(19) United States
(12) Patent Application Publication
(10) Pub. No.: US 2024/0002922 A1
(43) Pub. Date: Jan. 4, 2024

(54) METHODS FOR SIMULTANEOUS MOLECULAR AND SAMPLE BARCODING

(71) Applicant: GUARDANT HEALTH, INC., Palo Alto, CA (US)

(72) Inventor: Andrew KENNEDY, San Diego, CA (US)

(21) Appl. No.: 18/342,408

(22) Filed: Jun. 27, 2023

Related U.S. Application Data
(63) Continuation of application No. PCT/US22/41099, filed on Aug. 22, 2022.
(60) Provisional application No. 63/235,640, filed on Aug. 20, 2021.

(51) Int. Cl. C12Q 1/6855; C12Q 1/6806

(52) U.S. Cl. CPC C12Q 1/6855; C12Q 1/6806

(57) ABSTRACT

The present application provides methods of sequencing populations of nucleic acids within multiple pooled samples with tracking of individual molecules and their samples of origin. In such methods, the same sequencing read provides in line sequences of sample and molecular barcodes and a sample molecule allowing deconvolution of sequencing reads to sample of origin and grouping of amplification copies of original molecules into families. The methods are amenable to multiple sequencing platforms, reduce uninformative portions of sequencing reads on adapter sequence common to all adapters, decrease opportunity for labelling samples with the wrong barcode (index hopping), and provide additional multiplexing capacity.

### CROSS REFERENCE TO RELATED APPLICATION

This application is a continuation of International PCT Application No. PCT/US2022/041099, filed Aug. 22, 2022, which claims the benefit of 63/235,640, filed Aug. 20, 2021, both of which are incorporated by reference in its entirety for all purposes.

### BACKGROUND

A tumor is an abnormal growth of cells. Fragmented DNA is often released into bodily fluid when cells, such as tumor cells, die. Thus, some of the cell-free DNA in body fluids is tumor DNA. A tumor can be benign or malignant. A malignant tumor is often referred to as a cancer.

Cancer is a major cause of disease worldwide. Each year, tens of millions of people are diagnosed with cancer around the world, and more than half eventually die from it. In many countries, cancer ranks as the second most common cause of death following cardiovascular diseases. Early detection is associated with improved outcomes for many cancers.

Cancer is caused by the accumulation of mutations and/or epigenetic variations within an individual's normal cells, at least some of which result in improperly regulated cell division. Such mutations commonly include copy number variations (CNVs), copy number aberrations (CNA), single nucleotide variations (SNVs), gene fusions and indels, and epigenetic variations include modifications to the 5th atom of the 6-atom ring of cytosine and association of DNA with chromatin and transcription factors.

Cancers are often detected by biopsies of tumors followed by analysis of cells, markers or DNA extracted from cells. But more recently it has been proposed that cancers can also be detected from cell-free nucleic acids in body fluids, such as blood or urine (see, e.g., Siravegna et al., Nature Reviews Clinical Oncology 14, 531-548 (2017)). Such tests have the advantage that they are non-invasive and can be performed without identifying suspected cancer cells through biopsy. However, such tests are complicated by the fact that the amount of nucleic acids in body fluids is very low and the nucleic acids within them are diverse.

### SUMMARY

The invention provides methods of sequencing populations of DNA molecules in multiple samples. Such methods comprise:
(a) ligating a population of DNA molecules from a first sample to a first set of adapters, such that molecules of the population are flanked by an adapter on each side, wherein each adapter includes primer binding sites, and a molecular barcode varying among members of the set of adapters and a sample barcode that is the same among members of the set of adapters, wherein the molecular and sample barcodes are situated in the adapter such that a sequencing read initiating from one of the primer binding site of the adapter includes sequence of the sample and molecular barcodes followed by sequence of a DNA molecule of the first sample;
(b) repeating step (a) on populations of DNA molecules from one or more further samples, except that the populations of DNA molecules from each sample are ligated to different set of adapters, wherein the sample barcode varies among the different sets of adapters;
(c) amplifying the DNA molecules flanked by adapters to generate amplicons, each amplicon comprising a DNA molecule flanked by barcodes of the adapters on each side, flanked by primer binding sites of the adapters on each side;
(d) obtaining sequencing reads of the amplicons, wherein each sequencing read is initiated from one of the sequencing primer binding sites provided by the adapters; and
(e) segregating the sequence reads according to the sample of origin from a sample barcode portion of the reads and DNA molecule of origin from a molecular barcode portion of the reads to produce for each sample a plurality of families of sequencing reads, the families corresponding to different original molecules.

Some methods further comprise (f) calling out genetic variations, if present, for different samples from the plurality of families of sequencing reads for a sample. Step (f) can comprise for some or all of the families, calling out consensus nucleotides or consensus sequence in a family based on the sequencing reads in that family; and calling out genetic variations, if present, for each sample based on the consensus nucleotides and/or consensus sequences present in families for that sample.

Some method further comprise pooling the adapted DNA molecules from the different samples after step (b) before step (c). In some methods, step (c) is performed separately for different samples with a primer containing a pool index, and the method further comprises pooling amplification products after step (c).

In some methods, the same set of molecular barcodes is used for each set of adapters. In some methods, the sample barcode portion and the molecular barcode portion are contiguous sequences. In some methods, each adapter has two sample barcodes. In some methods, the sequencing reads in at least some of the families include sequencing reads of both strands of the same original molecule. In some methods, segregation into families is based on molecular barcode sequences and sequences of the molecules of the population. In some embodiments, the sequences of the molecules can include the start genomic position and stop genomic position of the molecule obtained from the sequencing reads. It can include the genomic start position of the sequencing read at which the 5′ end of the sequencing read is determined to start aligning to reference sequence and the genomic stop position of the sequencing read at which the 3′ end of the sequencing read is determined to stop aligning to the reference sequence. In some embodiments, the sequences of the molecules comprises (i) the first 1, first 2, the first 5, the first 10, the first 15, the first 20, the first 25, the first 30 or at least the first 30 base positions at the 5′ end of the sequencing read that align to the reference sequence, and/or (ii) the last 1, last 2, the last 5, the last 10, the last 15, the last 20, the last 25, the last 30 or at least the last 30 base positions at the 3′ end of the sequencing read that align to the reference sequence. In some methods, the adapters comprise one or more double-stranded portions and one or more single-stranded portions. In some methods, the adapters are Y-shaped adapters comprising two strands duplexed in a double-stranded portion and unduplexed in single-stranded portions. In some methods, the adapters are stem-loop adapters, the stem providing a double-stranded portion, and the loop comprising two single-stranded portions separated by a uracil or deoxyuridine residue. In some methods, the adapters are bubble adapters comprising two strands, forming unduplexed single-stranded portions flanked by duplexed double-stranded portions. In some methods, the primer binding sites are in the single-stranded portions of the adapters. In some methods, the molecular barcode of each adapter is in a double-stranded portion of the adapter. In some methods, the molecular barcode of each adapter is flush with the free end of the double-stranded portion of the adapter containing the molecular barcode portion. In some methods, the sample barcode and the molecular barcode are separate but contiguous sequences. In some methods, the sample barcode and the molecular barcode are separate but contiguous sequences within the double-stranded portion of the adapters. In some methods, the double-stranded portion of the adapters consists of the sample barcode and the molecular barcode. In some methods, the molecular barcode is in a double-stranded portion and the sample barcode or barcodes is within one or both of the single-stranded portions of the adapters. In some methods, the molecular barcode is in the double-stranded portion and two sample barcodes are respectively within the single stranded portions of the adapters.

In some methods, the DNA molecules are cell-free DNA molecules. In some methods, the molecular barcodes non-uniquely label the DNA molecules in the sample. In some methods, the number of different pairwise combinations of molecular barcodes is less than 1/104 of the number of DNA molecules. In some methods, the amplification is performed with primers binding to the primer binding sites.

The invention further provides methods of sequencing populations of DNA molecules in multiple samples. Such methods comprise:
(a) ligating a population of DNA molecules from a first sample to a first set of adapters, such that molecules of the population are flanked by an adapter on each side, wherein each adapter includes primer binding sites, and a barcode varying among members of the set of adapters, wherein the barcode is situated in the adapter such that a sequencing read initiating from one of the primer binding site of the adapter includes sequence of the barcode followed by sequence of a DNA molecule of the first sample;
(b) repeating step (a) on populations of DNA molecules from one or more further samples, except that the populations of DNA molecules from each sample are ligated to different set of adapters;
(c) amplifying the DNA molecules flanked by adapters to generate amplicons, each amplicon comprising a DNA molecule flanked by barcodes of the adapters on each side, flanked by primer binding sites of the adapters on each side;
(d) obtaining sequencing reads of the amplicons, wherein each sequencing read is initiated from one of the sequencing primer binding sites provided by the adapters; and
(e) segregating the sequence reads according to the sample of origin and DNA molecule of origin from a barcode portion of the reads to produce for each sample a plurality of families of sequencing reads, the families corresponding to different original molecules.

Some methods further comprise step (f): calling out genetic variations, if present, for different samples from the plurality of families of sequencing reads for a sample. In some methods, step (f) comprises for some or all of the families, calling out consensus nucleotides or consensus sequence in a family based on the sequencing reads in that family; and calling out genetic variations, if present, for each sample based on the consensus nucleotides and/or consensus sequences present in families for that sample.

Some methods further comprise pooling the adapted DNA molecules from the different samples after step (b) and before step (c). In some methods, step (c) is performed separately for different samples with a primer containing a pool index, and the method further comprises pooling amplification products after step (c). In some methods, the sequencing reads in at least some of the families include sequencing reads of both strands of the same original molecule. In some methods, segregation into families is based on barcode sequences and sequences of the molecules of the population. In some methods, the adapters comprise one or more double-stranded portions and one or more single-stranded portions. In some methods, the adapters are Y-shaped adapters comprising two strands duplexed in a double-stranded portion and unduplexed in single-stranded portions. In some methods, the adapters are stem-loop adapters, the stem providing a double-stranded portion, and the loop comprising two single-stranded portions separated by a uracil or deoxyuridine residue. In some methods, the adapters are bubble adapters comprising two strands, forming unduplexed single-stranded portions flanked by duplexed double-stranded portions. In some methods, the primer binding sites are in the single-stranded portions of the adapters.

The invention further provides a kit comprising (a) a first set of adapters comprising a sample barcode and a molecular barcode, wherein the sample barcode is the same in molecules of the first set and the molecular barcodes vary among a set of molecular barcodes among molecules of the first set; and (b) one or more further sets of adapters comprising a sample barcode and a molecular barcode, wherein the sample barcode is the same in molecules of the same set different than any other set in the kit, and the molecular barcodes vary among the set of molecular barcodes among member of each of the one or more sets. Optionally the adapters comprise one or more double-stranded portions and one or more single-stranded portions. Optionally, the adapters are Y-shaped adapters comprising two strands duplexed in a double-stranded portion and unduplexed in single-stranded portions. Optionally, the adapters are stem-loop adapters, the stem providing a double-stranded portion, and the loop comprising two single-stranded portions separated by a uracil or deoxyuridine residue. Optionally, the adapters are bubble adapters comprising two strands, forming unduplexed single-stranded portions flanked by duplexed double-stranded portions. Optionally, the molecular barcode of each adapter is in a double-stranded portion of the adapter. Optionally, the molecular barcode of each adapter is flush with the free end of the double-stranded portion of the adapter containing the molecular barcode portion. Optionally, the sample barcode and the molecular barcode are separate but contiguous sequences within the double-stranded portion of the adapters. Optionally, the double-stranded portion of the adapters consists of the sample barcode and the molecular barcode. Optionally, the molecular barcode is in a double-stranded portion and the sample barcode or sample barcodes is/are within one or both of the single-stranded portions of the adapters.

The invention further provide methods of sequencing populations of DNA molecules in multiple samples. Such methods comprise:
(a) ligating a population of DNA molecules from a first sample to a set of adapters comprising a double-stranded portion and single-stranded portions, such that molecules of the population are flanked by an adapter on each side, wherein each adapter in the set includes a double-stranded portion including a molecular barcode, a 3′ single-stranded portion including a first primer binding site adjacent a sample barcode universal binding site including unnatural bases and a 5′ single-stranded portion including a second primer binding site, and;
(b) repeating step (a) on populations of DNA molecules from one or more further samples;
(c) for each sample, amplifying the DNA molecules flanked by adapters with a primer pair comprising a forward primer containing a segment complementary to the first primer binding site and a sample barcode, the sample barcodes differing among the samples, and a reverse primer complementary to the second primer binding site to generate amplicons, wherein each amplicon comprises a DNA molecule from the samples, flanked by molecular barcodes from the adapters flanked by a sample barcode from the first primer;
(d) obtaining sequencing reads of the DNA molecules including molecular barcodes of the adapters and sample barcodes of the forward primers, wherein each sequencing read is initiated from a primer binding site from an adapter; and
(e) segregating the sequence reads according to the sample of origin from sequences of the sample barcodes and DNA molecule of origin from sequences of the molecular barcodes to produce for each sample a plurality of families of sequencing reads, the families corresponding to different original molecules.

Some methods further comprise (f) calling out genetic variations, if present, for different samples from the plurality of families of sequencing reads for a sample. Optionally step (f) comprises for some or all of the families, calling out consensus nucleotides or a consensus sequence in a family based on the sequencing reads in that family; and calling out genetic variations, if present, for each sample based on the consensus nucleotides and/or consensus sequences present in families for that sample. Optionally the adapters comprise one or more double-stranded portions and one or more single-stranded portions. Optionally, the adapters are Y-shaped adapters comprising two strands duplexed in a double-stranded portion and unduplexed in single-stranded portions. Optionally, the adapters are stem-loop adapters, the stem providing a double-stranded portion, and the loop comprising two single-stranded portions separated by a uracil or deoxyuridine residue. Optionally, the adapters are bubble adapters comprising two strands, forming unduplexed single-stranded portions flanked by duplexed double-stranded portions.

The invention further provides a kit comprising: (a) a set of adapters, wherein each adapter in the set include a double-stranded portion including a molecular barcode, a 3′ single-stranded portion including a forward primer binding site adjacent a universal sample barcode binding site including unnatural bases and a 5′ single stranded portion including a reverse primer binding site; (b) a set of primers, each primer of the set comprising a segment complementary to the forward primer binding site and a sample barcode, the sample barcodes differing among the primers; and (c) a primer complementary to the reverse primer binding site. Optionally, the adapters comprise one or more double-stranded portions and one or more single-stranded portions. Optionally, the unnatural bases are selected independently from nitroindole and deoxyinosine. Optionally, the adapters are Y-shaped adapters comprising two strands duplexed in a double-stranded portion and unduplexed in single-stranded portions. Optionally, the adapters are stem-loop adapters, the stem providing a double-stranded portion, and the loop comprising two single-stranded portions separated by a uracil or deoxyuridine residue. Optionally, the adapters are bubble adapters comprising two strands, forming unduplexed single-stranded portions flanked by duplexed double-stranded portions.

The invention further provide methods of generating a sequencing library, comprising ligating DNA molecules from a sample to a set of adapters, such that molecules of the population are flanked by an adapter on each side, wherein each adapter includes primer binding sites, and a sample barcode that is the same in members of the set and a molecular barcode varying among members of the set, wherein the sample and molecular barcodes are situated in the adapter such that a sequencing read initiating from one of the primer binding sites of the adapter includes sequence of sample and molecular barcodes followed by sequence of a DNA molecule from the sample. Some methods are for generating a plurality of sequencing libraries from a plurality of samples, further comprising repeating the ligating step on DNA molecules from one or more further samples, except that the DNA molecules from each sample are ligated to different set of adapters, the sample barcodes varying among the different sets of adapters. Optionally, the method further comprises amplifying the DNA molecules flanked by the adapters.

The invention further provides an adapter comprising a double-stranded portion and single-stranded portions, a molecular barcode, a sample barcode and primer binding sites, wherein the molecular barcode is situated in the double-stranded portion, the sample barcode is situated in the double-stranded portion or a single-stranded portion, and the primer binding sites are respectively situated in the single-stranded portions. Optionally, the adapter comprises two sample barcodes, one situated in each of the single-stranded portions.

The invention further provides methods of sequencing DNA populations in multiple samples. Such methods comprise:
(a) ligating a population of DNA molecules from a first sample to a first set of adapters, such that molecules of the population are flanked by an adapter on each side, wherein each adapter includes primer binding sites, and a barcode varying among members of the set of adapters, wherein the barcode is situated in the adapter such that a sequencing read initiating from one of the primer binding site of the adapter includes sequence of the barcode followed by sequence of a DNA molecule of the first sample;
(b) repeating step (a) on populations of DNA molecules from one or more further samples, except that the populations of DNA molecules from each sample are ligated to different set of adapters;
(c) amplifying the DNA molecules flanked by adapters to generate amplicons, each amplicon comprising a DNA molecule flanked by barcodes of the adapters on each side, flanked by primer binding sites of the adapters on each side;
(d) obtaining sequencing reads of the amplicons, wherein each sequencing read is initiated from one of the sequencing primer binding sites provided by the adapters; and
(e) segregating the sequence reads according to the sample of origin and DNA molecule of origin from a barcode portion of the reads to produce for each sample a plurality of families of sequencing reads, the families corresponding to different original molecules. Some methods further comprise (f) calling out genetic variations, if present, for different samples from the plurality of families of sequencing reads for a sample. In some methods, the barcode in each adapter has a sample barcode portion and a molecular barcode portion, wherein adapters within the same set have the same sample barcode, and adapters in different sets have different sample barcodes, and the molecular barcodes vary among a common set of molecular barcode in each set of adapters.

### BRIEF DESCRIPTION OF THE FIGURES

FIG. 1 shows formation of a library for sequencing using Y-shaped adapters containing sample and molecular barcodes (ILMN=Illumina).

FIG. 2 shows formation of a library using adapters as in FIG. 1 with additional multiplexing provided by including further sample barcodes in amplification primers.

FIG. 3 shows a comparison of three formats. The left hand format is a reference format in which adapters include only a molecular barcode. The center format shows adapters with separate sample and molecular barcodes. The right hand format shows adapters including a single barcode that serves as both a molecular and sample barcode.

FIG. 4 shows a further format in which a Y-shaped adapter includes a molecular barcode in its double-stranded portion and a universal primer binding site formed of unnatural nucleotides in a single-stranded portion to allow introduction of a sample barcode contiguous with the molecular barcode in a subsequent amplification step.

FIG. 5 shows exemplary adapters used for analyzing two samples.

FIGS. 6A, B shows sequences reads from samples 1 and 2 respectively aligned against the human genome.

### DEFINITIONS

A subject refers to an animal, such as a mammalian species (preferably human) or avian (e.g., bird) species, or other organism, such as a plant. More specifically, a subject can be a vertebrate, e.g., a mammal such as a mouse, a primate, a simian or a human. Animals include farm animals, sport animals, and pets. A subject can be a healthy individual, an individual that has symptoms or signs or is suspected of having a disease or a predisposition to the disease, or an individual that is in need of therapy or suspected of needing therapy.

A genetic variation refers to a change in nucleotide sequence (nucleotide variation), modification, or copy number relative to that of a reference sequence, which can be e.g., an exon, gene, chromosome or full genome representing the normal sequence, modification, if any, and copy number for an organism. A genetic variation can include one or more single nucleotide variations (SNVs), insertions, deletions, repeats, small insertions, small deletions, small repeats, structural variant junctions, variable length tandem repeats, and/or flanking sequences, copy number variants (CNVs), transversions, gene fusions and other rearrangements, as well as modifications such as methylation, acetylation or hydroxymethylation are also forms of genetic variation. A variation can be a base change, insertion, deletion, repeat, copy number variation, modification, transversion, or any combination thereof.

A cancer marker is a genetic variation associated with presence or risk of developing a cancer. A cancer marker can provide an indication a subject has cancer or a higher risk of developing cancer than an age and gender matched subject of the same species that does not have the cancer marker. A cancer marker may or may not be causative of cancer.

The four standard nucleotide types refer to A, C, G, T for deoxyribonucleotides and A, C, T and U for ribonucleotides.

Within a sequencing read the terms “upstream” and “downstream” are used to indicate sequences relatively closer or further to the point of initiation of sequencing, typically a sequencing primer binding site. For example, if a sequencing read includes an upstream and downstream molecular barcode, the upstream molecular barcode is closer than the downstream molecular barcode to the point of initiation of sequencing.

A forward primer is a primer initiating first strand synthesis from an adapter, and a reverse primer is a primer initiating second strand synthesis.

Unless otherwise apparent from the context, reference to a nucleic acid can include DNA or RNA. Nucleic acid molecules isolated from nature typically contain standard nucleotides, including naturally modified forms thereof, such as methylcytosine. Synthetic oligonucleotides, such as adapters, can also be formed entirely from these standard nucleotides, or can include, one or more positions occupied by analogs of these standard nucleotides, capable of base pairing with one, some or all of the standard nucleotides. Nitroindole and deoxyinosine are examples of analog nucleotides capable of pairing with any of the standard nucleotides. Some synthetic oligonucleotides, such as adapters, are formed entirely of standard nucleotides of DNA. Some synthetic oligonucleotides, such as a adapters, include uracil or deoxyuridine as well as standard DNA nucleotides. Analogs including nitroindole and deoxyinosine can also be referred to as unnatural bases.

### DETAILED DESCRIPTION

### I. General

The present application provides methods of sequencing populations of nucleic acids within multiple pooled samples with tracking of individual molecules and their samples of origin. In such methods, the same sequencing read provides in-line sequences of sample and molecular barcodes and a sample molecule allowing deconvolution of sequencing reads to sample of origin and grouping of amplification copies of original molecules into families. The methods are amenable to multiple sequencing platforms, reduce uninformative portions of sequencing reads on adapter sequence common to all adapters, decrease opportunity for labelling samples with the wrong sample barcode (index hopping), and provide additional multiplexing capacity.

### II. Sample and Molecular Barcodes and Adapters

A barcode is a short nucleic acid (e.g., less than 500, 100, 50, 20, 15, 10 or 5 nucleotides long), used to label nucleic acid molecules to distinguish nucleic acids from different samples (a sample barcode), or different nucleic acid molecules in the same sample (a molecular barcode) or the same barcode can be used to distinguish both samples and molecules within samples. Sample and molecular barcodes can be referred to collectively simply as barcodes. Thus reference to a barcode can indicate a barcode that serves both as sample and molecular barcodes. Alternatively, it can indicate a barcode having separate sample and molecular barcode portions. The particular code stored by a barcode can be referred to as a designation of a barcode.

Barcodes are typically provided as sets of multiple different individual barcodes for distinguishing samples and molecules or both. That is, different samples receive different sample barcodes from a set of sample barcodes, and different molecules within a sample receive different molecular barcodes from a set of molecular barcodes. Barcodes can be single-stranded, double-stranded or have both single and double-stranded components. If a double-stranded component is present, the strands can be of the same or unequal lengths. Barcodes can have the same or different lengths within a set. Barcodes can be random, non-random or semi-random sequences in which at least one position is randomly selected and at least one is not. Barcodes can be synthesized together with pooling of nucleotides at random positions, or individually. Some sets of barcodes having sequences selected such that there is a Hamming distance of at least 2, 3, 4 or 5 nucleotides between each barcode in a set. Barcodes can also be selected to avoid sequences that hybridize within one another or other molecules within a reaction, to avoid sequences subject to sequencing errors, or sequences subject to confusion with sequences of other barcodes. Barcodes as components of adapters or tails of amplification primers can be attached to one end or both ends of nucleic acids to be labelled.

Sample barcodes can be decoded to reveal sample of origin. Sample barcodes allowing pooling and parallel processing of multiple samples after the barcodes have been attached. The number of a different sample barcodes within a set is typically sufficient that each different sample is associated with a different sample barcode or combination of barcodes. Alternatively, samples can be divided into subsets with samples in a subset receiving the same sample barcode and samples in different subsets receiving different sample barcodes.

Molecular barcodes are used to track original molecules within the same sample. They can be decoded to reveal amplification copies or sequencing reads thereof of the same original molecule. The number of molecular barcodes within a set or number of pairwise combinations within a set if sample molecules are labelled with molecular barcodes from both ends can be sufficient such that there is a high probability (e.g., at least 80, 90, 95 or 99% probability) that substantially all original molecules in sample that complete ligation with an adapter or pair of adapters (e.g., at least 75%, 90%, 95% or 99%) receives a different molecular barcode or different combination of molecular barcodes (unique barcoding). Alternatively, the number of molecular barcodes or pairwise combinations of molecular barcodes can be substantially less than the number of molecules within a sample, e.g., a ratio of different molecular barcodes or pairwise combination of molecular barcodes to samples molecules of less than 1:10³, 1:10⁴, 1:10⁵, 1-10⁶, 1:10⁷, 1-10⁸, 1:10⁹, 1:10¹⁰, 1:10¹¹ or 1:10¹² (non-unique barcoding). In this case, multiples molecules within the same sample receive the same molecular barcode or combination of molecular barcodes. However, amplification products of the same original molecule or their sequencing reads can still be distinguished by using a combination of the molecular barcodes and information from the sequencing reads, such as the start and stop points (i.e., genomic start position of the sequencing read at which the 5′ end of the sequencing read is determined to start aligning to reference sequence and genomic stop position of the sequencing read at which the 3′ end of the sequencing read is determined to stop aligning to the reference sequence) or length of sequencing reads. In some embodiments, the information from the sequencing reads comprises: (i) the first 1, first 2, the first 5, the first 10, the first 15, the first 20, the first 25, the first 30 or at least the first 30 base positions at the 5′ end of the sequencing read that align to the reference sequence; and/or (ii) the last 1, last 2, the last 5, the last 10, the last 15, the last 20, the last 25, the last 30 or at least the last 30 base positions at the 3′ end of the sequencing read that align to the reference sequence. Typically sufficient different molecular barcodes or combinations of molecular barcodes are used such that there is high probability (e.g., at least 90%, at least 95%, at least 98%, at least 99%, at least 99.9% or at least 99.99%) that all nucleic acids mapping to a particular genomic region defined by same start and stop points bear a different molecular barcode. Generally, assignment of unique or non-unique molecular barcodes in reactions follows methods and systems described by US patent applications 20010053519, 20030152490, 20110160078, and U.S. Pat. Nos. 6,582,908 and 7,537,898.

In some cases, the number of different molecular barcodes is at least 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000. In other cases, the number of different molecular barcodes is less than 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique identifiers per genome sample. The number of different molecular barcodes in a set depends on whether unique or nonunique barcoding is used and whether molecular barcodes are used to label nucleic acid sample molecules individually or in pairwise combinations. Other things being equal, more different molecular barcodes are needed for unique than non-unique labelling. Also more different molecular barcodes are needed for labelling with individual molecular barcodes per sample nucleic acid than in pairwise combinations, because the number of combinations is the square of the number of individual labels.

The number of different molecular barcodes necessary for unique labelling of nucleic molecules is a function of how many original nucleic acid molecules are in the sample or part thereof being analyzed. This, in turn, depends on such factors at the total number of haploid genome equivalents in the sample, the average and variance in size of nucleic acid molecules, and the ligation efficiency of adapters including barcodes.

For non-unique barcoding the number of molecular barcode combinations (square of number of different molecular barcodes) is sometimes least any of 64, 100, 400, 900, 1400, 2500, 5625, 10,000, 14,400, 22,500 or 40,000 and no more than any of 90,000, 40,000, 22,500, 14,400 or 10,000. For example, the number of barcode combinations can be between 64 and between 400 and 22,500, 400 and 14,400 or between 900 and 14,400. The number of different molecular barcode combinations (n) can be between 2 and 100,000*z, wherein z is a measure of central tendency (e.g., mean, median, mode) of an expected number of duplicate molecules having the same start and stop positions. The number of different molecular barcode combinations can be at least any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit). Optionally, n is no greater than 100,000*z, 10,000*z, 2000*z, 1000*z, 500*z or 100*z (e.g., upper limit). Thus, n can range between any combination of these lower and upper limits. The number of combinations can be between 100*z and 1000*z, 5*z and 15*z, between 8*z and 12*z, or about 10*z. For example, a haploid human genome equivalent has about 3 picograms of DNA. A sample of about 1 microgram of DNA contains about 300,000 haploid human genome equivalents. The number n can be between 15 and 45, between 24 and 36, between 64 and 2500, between 625 and 31,000, or about 900 and 4000. For example, a sample comprising about 10,000 haploid human genome equivalents of cfDNA can be barcoded with about 36 combinations of six different molecular barcodes. Samples barcoded in such a way can be those with a range of about 10 ng to any of about 100 ng, about 1 about 10 μg of fragmented polynucleotides, e.g., genomic DNA, e.g. cfDNA.

Adapters are relatively short nucleic acids for attachment to the ends of sample molecules to facilitate amplification, sequencing and tracking of the sample molecules. The total length of each adaptor (measured by the longest strand if more than one) is e.g., less than 250, 150, 100, 75 or 50 nucleotides long. The free end of the double-stranded portion serves for joining of a sample molecule (e.g., by blunt or cohesive end ligation). Adapters can include the sample and molecular barcodes discussed above. Adapters can include primer binding sites to permit binding of amplification primers for amplification of a nucleic acid molecule flanked by adapters at both ends, and/or sequencing primers for generating a sequence read. Adapters can also include binding sites for capture probes, such as an oligonucleotide attached to a flow cell support.

Some adapters have one or more double-stranded portions and one or more single-stranded portions. Y-shaped adapters (see, e.g., U.S. Pat. No. 7,741,463), stem-loop (see e.g., U.S. Pat. No. 10,155,939) and bubble adapters (see US20180030532A1) are examples of such adapters. Y-shaped adapters are nucleic acids formed from two strands, which are paired in a double-stranded portion (with the possible exception of a single-stranded overhang to facilitate ligation), and also unpaired in single-stranded portions. The two single-stranded portions can be represented in the shape of the letter V joined to the double-stranded portion, together forming a Y-shape. Y-shaped adapters have one free end in the double-stranded portion, which can be a blunt end or an end in which one strand overhangs the other, e.g., by a single nucleotide. Each of the unpaired single strands has a single-stranded end. The total length of each strand of Y-shaped adapters is e.g., less than 250, 150, 100, 75 or 50 nucleotides long. A standard Illumina Y-shaped adapter without sample or molecular barcodes has a strand length of about 115 nucleotides. The free end of the double-stranded portion serves for joining of a sample molecule (e.g., by blunt or cohesive end ligation).

Stem-loop adapters (e.g., NebNext from New England Biolabs) are similar to Y-shaped adapters except that the single-stranded portions are joined via a uracil residue thus forming a loop instead of a V. Thus, stem-loop adapters are a single strand with a duplexed stem corresponding to the double-stranded portion of Y-shaped adapters, and a loop including two single-stranded portions of DNA separated by a uracil (U) or deoxyuridine (dU), which correspond to the single-stranded portions of Y-shaped adapters. The residues immediately adjacent the U or dU are the single-stranded-end residues of the single-stranded portions in stem-loop adapters. The stem has a free end that can be blunt or tailed as in the stem of Y-shaped adapters and is used for joining to a sample molecule. After joining of stem-loop adapters to a sample molecule, the U or dU can be enzymatically removed leaving the same topography as for Y-shaped adapters. USER Enzyme from NEB is a mixture of Uracil DNA glycosylase (UDG) and the DNA glycosylase-lyase Endonuclease VIII (DGLE). UDG catalyzes the excision of a uracil or deoxyuridine base, forming an abasic (apyrimidinic) site while leaving the phosphodiester backbone intact, and DGLE removes the abasic nucleotide.

Bubble adapters (BGI) are similar to stem-loop adapters and Y-shaped adapters except that the V-region of Y-shaped adapter or the loop of stem-loop adapters is replaced by a bubble of two unduplexed single stranded portions flanked on both sides by double-stranded portions. Bubble adapters typically have two strands of unequal length with some or all of the length difference being in the single-stranded portions. The 5′ end of the longer nucleic acid has a phosphorylated nucleotide. The 3′ end of the shorter nucleic acid typically has an overhang from the end of an otherwise double-stranded portion. The double-stranded portion containing the phosphorylated 5′ nucleotide and overhang if present corresponds with the stem of stem-loop adapters or the double-stranded portion of Y-shaped adapters, and ligates with a sample nucleic acid molecule. This double-stranded portion can be referred to as the downstream double-stranded portion because it provides the site of ligation to a sample molecule. The other double-stranded portion can be referred to an upstream double-stranded portion because it is further from the sample molecule. The two single-strands in the middle forming a bubble correspond with the single-stranded portions forming a V in Y-shaped adapters or the single-stranded portions separated by a uracil or deoxyuridine in stem-loop adapters. Bubble adapters can include a U or dU in the shorter strand, longer strand or both to separate the single-stranded portions from the upstream double-stranded portion. Usually such a U or dU is included in the longer strand. The U or dU can be excised as with stem-loop adapters after ligation of the adapters to sample molecules leaving adapters in a Y-shape.

Although much of the exemplification that follows is based on Y-shaped adapters for ease of illustration the same formats apply to stem-loop and bubble adapters or other adapters with corresponding topological features.

Adapters can include the sample and molecular barcodes discussed above. Adapters can include primer binding sites to permit binding of amplification primers for amplification of a nucleic acid molecule flanked by adapters at both ends, and/or sequencing primers for generating a sequence read. Primer binding sites are typically provided in the single-stranded portions of a Y-shaped, stem-loop or bubble adapter. The asymmetry of unpaired single-stranded portions allows strand-specific sequencing from two primers binding to the respective single strands. Adapters can also include binding sites for capture probes, such as an oligonucleotide attached to a flow cell support.

Sample and molecular barcodes can be separated and contiguous with one another, separated with an intervening nucleotide or sequence of nucleotides between them, or can be encoded within the same sequence. If intervening nucleotides are present, the number of intervening nucleotides can be less than 20, 15, 10, 5, 4, 3, or 2. Reduction of the number of intervening nucleotides is advantageous in maximizing the proportion of a sequencing read available for the sample molecule

In one format, sample and molecular barcodes are separate and contiguous with both in the double-stranded portion of a Y-shaped, stem-loop or bubble adapter with the molecular barcode at (i.e., co-terminal or flush with) or closer to the double-stranded end of the adapter, and the sample barcode between the molecular barcode and the single-stranded ends of the adapter. The double-stranded portion of such adapters can be blunt-ended or can have a single stranded overhang (e.g., single nucleotide T) to facilitate annealing. If such an overhang is present, the molecular barcode is considered co-terminal or flush with the end of the double-stranded portion when the molecular barcode is coextensive with the double-stranded portion (i.e., ignoring the single-stranded overhang). Such an arrangement allows a sequencing read initiated from a primer binding site in a single stranded portion of the adapter to include sequence of an upstream sample barcode followed by an upstream molecular barcode followed by a sample nucleic acid molecule followed by a downstream molecular barcode followed by a downstream sample barcode, which is often the same as the upstream sample barcode and does not therefore need to be read. Optionally, the double-stranded portion of such adapter (not including a single-stranded overhang if present to facilitate ligation) consists of a molecular barcode and a sample barcode. The positions of molecular and sample barcodes can also be reversed to generate a sequencing read comprising first molecular barcode, first sample barcode, sample nucleic acid molecule, second sample barcode, and second molecular barcode. In another format, the molecular barcode is in a double-stranded portion of a Y-shaped, stem-loop or bubble adapter, and the sample barcode is in a single-stranded portion. In another format, the molecular barcode is in a double-stranded portion of a Y-shaped, stem-loop or bubble adapter, and two sample barcodes are in respective single-stranded portions. Such a topology allows generation of sequencing reads containing different upstream and downstream sample barcodes and sample identification based on the combination of the two barcodes thus increasing multiplexing capacity. Optionally, a sample and a molecular barcode are immediately adjacent to each other (i.e., no intervening nucleotides) and the molecular barcode is co-terminal (i.e., flush) with the free end of a double-stranded portion of the Y-shaped, stem-loop or bubble adapter. A sequencing read initiated in a single-stranded portion containing the sample barcode upstream of the molecular barcode includes the sample barcode followed by an upstream molecular barcode followed by a downstream molecular barcode.

Contiguity of sample and molecular barcodes avoids expending part of the sequencing read on intervening nucleotides leaving more of the finite length of the sequencing read for the sample nucleic acid molecule sequences. Likewise, juxtaposing the molecular barcode with the double-stranded end of a Y-shaped, stem-loop or bubble adapter leaves more the sequencing read for sample nucleic acid molecule sequences. There is a balance between use of longer sequences to provide more permutations of sample and molecular barcodes and greater selection among the available permutations and shorter sequences to minimize the part of sequencing reads taken up by non-sample molecules. In some adapters, the sample and molecular barcodes each occupy 3-10 nucleotides. In some adapters, the combination of sample and molecular barcodes occupies 6-10 nucleotides, optionally 7 nucleotides.

The same or different adapters can be linked to the respective ends of a nucleic acid molecule. Usually the same adapter is linked to the respective ends except that the barcode is different. The sequences of adapters and particularly the segments for primer binding attachment to a flow cell can vary depending on the sequencing platform employed.

### III. Preparation of Sample Nucleic Acids

The methods are performed on a plurality of initially separate samples of nucleic acid. The samples can be obtained from different subjects, or the same subject at different times or from different sources (i.e., tissues or fluids) in the same subject. The samples undergo separate preparation and processing at least up to the point at which sample barcodes are attached.

A different set of adapters is typically used for different nucleic acid samples. Typically the different sets differ only in the barcodes from one another. If separate sample and molecular barcodes are used, then the adapters used for different sample can differ from one another only in the sample barcodes. For example, each sample can receive an adapter set, which has one sample barcode varying among the adapter sets, and a set of molecular barcodes, which is the same for the adapter sets. Thus, sample molecules from the same sample receive the same sample barcode and varying molecular barcodes. Sample molecule from a different sample receive a different sample barcode but may receive the same set of molecular barcodes. If sample and molecular barcodes are combined into a combined barcode, then a different set of combined barcodes can be used for each sample to be differentially labelled. The molecules in a particular sample receive a barcode or combination of barcodes that differs among molecules within the sample, and also differs from the barcodes linked to sample molecules in different samples. Typically, the set of such barcodes used for one sample is mutually exclusive with the set of barcodes used for any other sample. In other words, there are no barcodes commonly received by multiple samples.

Typically a sample molecule is ligated to an adapter at each end. Thus, if an adapter includes separate sample and molecular barcodes, flanking a sample molecule with an adapter at each end results in the sample molecule being flanked by two sample barcodes and two molecular barcodes. The two samples barcodes are typically the same as one another because a single sample barcode is sufficient to distinguish all molecules of one sample, from molecules of another sample receiving a different sample label. The two molecular barcodes can typically include any pairwise combination of the individual molecular barcodes in the set of molecular barcodes used to label any particular sample. If such a set contains n molecular barcodes, then there are n squared such combinations. As previously noted, the number of such combinations can exceed the number of molecules in a sample such that there is a high probability that each sample molecule receives a different combination of molecular barcodes. Or the number of such combinations can be less than the number of molecules, sometimes orders of magnitude less (non-unique barcoding).

If an adapter set includes a combined barcode to track samples and molecules, then ligation of a sample molecule to adapters at each end results in the molecule being flanked by two combined barcodes. As previously described for molecular barcodes, the two combined barcodes can include any combination of individual combined barcodes present in a set of adapters used for a particular sample.

After ligation of sample molecules to adapters including sample and molecular barcodes, the samples can be pooled and processed together with eventual deconvolution of sequencing reads to their sample of origin from the sample barcodes.

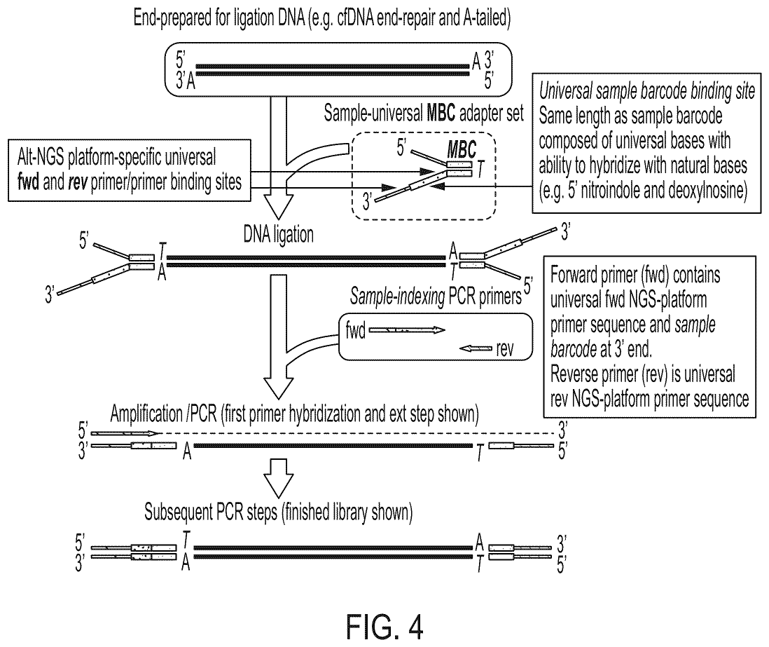

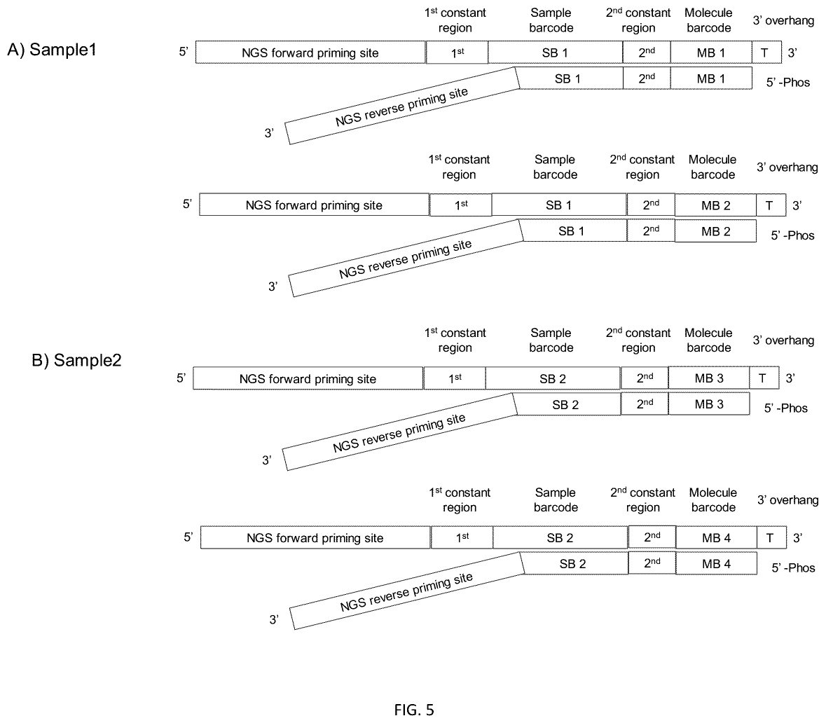

In a further variation, molecular barcodes are combined with a universal binding site for sample barcodes in the same adapter. The universal binding site is formed from nucleotides with unnatural bases, such as nitroindole (e.g., 5-nitroindole) and/or deoxyinosine that are able to duplex with any of the standard nucleotides (DNA or RNA). Such an adapter is configured to allow introduction of sample barcodes at a subsequent amplification step. An exemplary adapter includes a molecular barcode in a double-portion, and a universal binding site for sample barcodes in a single-stranded portion. Single-stranded portions of such adapters also include primer binding sites. A primer binding site can be adjacent to the universal binding site in an orientation as shown in FIG. 4. Optionally, there are no intervening nucleotides between the adjacent primer binding site and universal binding stie. Because sample barcodes are introduced after ligating such adapters to sample molecules, the same set of adapters can be used for any sample. The adapters in such a set typically differ only in their molecular barcodes.

In the above variation, adapters are ligated to populations of sample nucleic acids from multiple samples with the samples kept separate. An amplification reaction is then performed on the separate samples with a pair of forward and reverse primers. The forward primer contains a segment complementary to the first primer binding site and a sample barcode. This primer can duplex with a single-stranded portion of an adapter containing the first primer binding site and universal binding site, the sample barcode duplexing with the universal binding site. The sample barcodes differ in amplifications conducted for different samples so each sample receives a different sample barcodes. The reverse primer is complementary to the second primer binding site. Amplification generates amplicons comprising a sample nucleic acid flanked by molecular barcodes from the adapters flanked by sample barcodes from the forward primer. These amplicons now labelled with sample barcodes can be processed subsequently as for amplicons generated from adapters containing both molecular and sample barcodes.

A sample can be any biological sample isolated from a subject. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids.

The number of different samples can be greater than or equal to 2, 5, 10, 50, 100, 500, 1000, 2000, 5000, or 10,000. The volume of plasma can depend on the desired read depth for sequenced regions. Exemplary volumes are 0.4-40 mL, 5-20 mL, 10-20 mL. For examples, the volume can be 0.5 mL, 1 mL, 5 mL 10 mL, 20 mL, 30 mL, or 40 mL. A volume of sampled plasma may be for example 5 to 20 mL.

A sample can comprise various amount of nucleic acid that contains genome equivalents. For example, a sample of about 30 ng DNA can contain about 10,000 haploid human genome equivalents and, in the case of cell-free DNA, about 200 billion individual nucleic acid molecules. Similarly, a sample of about 100 ng of DNA can contain about 30,000 haploid human genome equivalents and, in the case of cell-free DNA, about 600 billion individual molecules. Some samples contain 1-500, 2-100, 5-150 ng cell-free DNA, e.g., 5-30 ng, or 10-150 ng cell-free DNA.

cfDNA has a peak of fragments at about 160 nucleotides (e.g., 168 nucleotides), and most of the fragments in this peak range from about 140 nucleotides to 180 nucleotides. Accordingly, cfDNA from a genome of about 3 billion bases (e.g., the human genome) may be comprised of almost 20 million (2×10⁷) polynucleotide fragments. A sample of about 30 ng DNA can contain about 10,000 haploid human genome equivalents. (Similarly, a sample of about 100 ng of DNA can contain about 30,000 haploid human genome equivalents.) A sample containing about 10,000 (104) haploid genome equivalents of such DNA can have about 200 billion (2×1011) individual polynucleotide molecules. It has been empirically determined that in a sample of about 10,000 haploid genome equivalents of human DNA, there are about 3 duplicate polynucleotides beginning at any given position. Thus, such a collection can contain a diversity of about 6×10¹⁰-8×10¹⁰ (about 60 billion-80 billion e.g., about 70 billion (7×10¹⁰)) differently sequenced polynucleotide molecules.

A sample can comprise nucleic acids of different types and origins. A sample can contains DNA or RNA or both. Nucleic acids can be single-stranded or double-stranded or be partly double-stranded and partly single-stranded. A sample can comprise germline DNA or somatic DNA or both. Nucleic acids within a sample can carry genetic variations, which can be carrying germline mutations and/or somatic mutations. Some such mutations can be cancer markers (e.g., cancer-associated somatic mutations).

Exemplary amounts of cell-free nucleic acids in a sample before amplification range from about 1 fg to about 1 ug, e.g., 1 pg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 pg, 100 pg, 1 ng, 10 ng, 100 ng, 150 ng, or 200 ng of cell-free nucleic acid molecules. The method can comprise obtaining 1 femtogram (fg) to 200 ng.

An exemplary sample is 5-10 ml of whole blood, plasma or serum, which includes about 30 ng of DNA or about 10,000 haploid genome equivalents.

Some samples contain cell-free nucleic acids. Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. Double-stranded DNA molecules at least some of which have single-stranded overhangs are a preferred form of cell-free DNA for any method disclosed herein. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells.

A cell-free nucleic acid can have one or more epigenetic modifications, for example, a cell-free nucleic acid can be acetylated, methylated, ubiquitinylated, phosphorylated, sumoylated, ribosylated, and/or citrullinated.

Cell-free nucleic acids have a size distribution of about 100-500 nucleotides, particularly 110 to about 230 nucleotides, with a mode of about 168 nucleotides and a second minor peak in a range between 240 to 440 nucleotides

Cell-free nucleic acids can be isolated from bodily fluids through a partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica based columns to remove contaminants or salts. Non-specific bulk carrier nucleic acids, for example, may be added throughout the reaction to optimize certain aspects of the procedure such as yield.

After such processing, samples can include various forms of nucleic acid including double-stranded DNA, single-stranded DNA and single-stranded RNA. Optionally, single-stranded DNA and RNA can be converted to double-stranded forms so they are included in subsequent processing and analysis steps.

Nucleic acid present in a sample with or without prior processing as described above typically contain a substantial portion of molecules in the form of partially double-stranded molecules with single-stranded overhangs. Such molecules can be converted to blunt-ended double-stranded molecules by treating with one or more enzymes to provide a 5′-3′ polymerase and a 3′-5′ exonuclease (or proof reading function), in the presence of all four standard nucleotide types. Such a combination of activities can extend strands with a recessed 3′ end so they end flush with the 5′ end of the opposing strand (in other words generating a blunt end) or can digest strands with 3′ overhangs so they are likewise flush with the 5′ end of the opposing strand. Both activities can optionally be conferred by a single polymerase. The polymerase is preferably heat-sensitive so that its activity can be terminated when the temperature is raised. Klenow large fragment and T4 polymerase are examples of suitable polymerase.

The resulting blunt-ended nucleic acids can be ligated to adapters with a double-stranded blunt free end or can be subject to tailing to generate cohesive ends, which pair with corresponding single-stranded overhangs at a double-stranded free end of adapters. Tailing of blunt ends can be by a polymerase lacking a proof reading function. This polymerase is preferably thermostabile such as to remain active at the elevated temperature that denatures the polymerase use for blunt ending. Taq, Bst large fragment and Tth polymerases are examples of such a polymerase. The second polymerase effects a non-templated addition of a single nucleotide to the 3′ ends of blunt-ended nucleic acids. Although the reaction mixture typically contains equal molar amounts of each of the four standard nucleotide types from the prior step, the four nucleotide types are not added to the 3′ ends in equal proportions. Rather A is added most frequently, followed by G followed by C and T. Such tailed molecules can be ligated to adapters with a complementary T or C overhand at the free end of the double-stranded portion.

Preferably, the present methods result in at least 75, 80, 85, 90 or 95% of double-stranded nucleic acids in the sample being linked to adapters. Preferably, the present methods result in at least 75, 80, 85, 90 or 95% of available double-stranded molecules in the sample being sequenced.

### IV. Amplification

Sample nucleic acids flanked by adapters can be amplified by PCR and other amplification methods typically primed from primers binding to primer binding sites in adapters flanking a nucleic acid to be amplified. Amplification methods can involve cycles of extension, denaturation and annealing resulting from thermocycling or can be isothermal as in transcription mediated amplification. Other amplification methods include the ligase chain reaction, strand displacement amplification, nucleic acid sequence based amplification, and self-sustained sequence based replication. Amplification can be performed once or multiple times.

Amplification can be performed before and distinct from sequencing or integrated with sequencing or both. Amplification can also be performed before or after enrichment of selected sample molecules, or both.

### V. Enrichment

Sample molecules can be subject to enrichment for sequences of interest. Enrichment can be performed by affinity purification, e.g., by hybridization to immobilized oligonucleotides complementary to the sequences of interest. Enrichment can be performed before or after ligation to adapters, and before or after amplification, or any combination thereof. If enrichment is performed before attachment of sample barcodes, the samples are enriched separately, whereas if enrichment is performed after attachment of sample barcodes it can be performed on pooled samples.

### VI. Sequencing

Sample nucleic acids flanked by adapters with or without prior amplification can be subject to sequencing. Sequencing methods preferably provide sequencing reads of sufficient length to tread through sample molecules and barcode sequences on one or both sides of a sample molecule in a single read. Sequencing methods include, for example, Sanger sequencing, high-throughput sequencing, pyrosequencing, sequencing-by-synthesis, single-molecule sequencing, single molecule real time sequencing (Pac-Bio), ONT-sequencing, exon sequencing, electron microscopy-based sequencing, panel sequencing, transistor-mediated sequencing direct sequencing, random shotgun sequencing, whole genome sequencing, capillary electrophoreses, gel electrophoresis, duplex sequencing, cycle sequencing, single-base extension sequencing, emulsion PCR, co-amplification at lower denaturation temperature-PCT (COLD-PCR), sequencing by reversible dye terminator, paired-end sequencing, near-term sequencing, exonuclease sequencing, sequencing by ligation, short-read sequencing, nanopore sequencing, semiconductor sequencing, sequencing-by-ligation, sequencing-by-hybridization, RNA-Seq (Illumina), Digital Gene Expression (Helicos), next generation sequencing, single molecule sequencing by synthesis (SMSS) (Helicos), massively-parallel sequencing, 454 sequencing, Clonal Single Molecule Array (Solexa/Illumina), shotgun sequencing, Ion Torrent, Oxford Nanopore, Roche Genia, Maxim-Gilbert sequencing, primer walking, SOLiD, Ion Torrent, MS-PET sequencing or Nanopore platforms, and combinations thereof. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

Sequencing reactions can be performed on sample nucleic acids molecules that have undergone amplification in the previous step. The sequence reactions may provide for sequence coverage of the genome of at least 5%, 10%, 15%, 20%, 25%, 30%, 40%, 50%, 60%, 70%, 80%, 90%, 95%, 99%, 99.9% or 100%. In other cases, sequence coverage of the genome may be less than 5%, 10%, 15%, 20%, 25%, 30%, 40%, 50%, 60%, 70%, 80%, 90%, 95%, 99%, 99.9% or 100%.

Simultaneous sequencing reactions may be performed using multiplex sequencing. In some cases, amplicons of sample nucleic acids may be sequenced with at least 1000, 2000, 3000, 4000, 5000, 6000, 7000, 8000, 9000, 10000, 50000, 100,000 sequencing reactions. In other cases, amplicons of sample nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions.

The sequencing method can be massively parallel sequencing, that is, simultaneously (or in rapid succession) sequencing any of at least 100, 1000, 10,000, 100,000, 1 million, 10 million, 100 million, or 1 billion nucleic acid molecules.

Sequencing can be performed in a single or paired read format with sample and molecular barcodes at least at the start of a read, and sometimes at the end of a read as well.

Samples can be split into two or more aliquots before or after pooling of samples for analysis of DNA modification (see, e.g., Gouil et al., Essays Biochem. 63(6):639-648 (2019)). One aliquot of samples is treated such that unmodified nucleotides undergo substitution by a different nucleotide. For example, in sodium bisulfite sequencing unmodified cytosines can be converted to uracil, whereas methylated cytosines are unmodified. Comparison of sequencing reads from the different aliquots indicates, which cytosines were subject of modification.

### VII. Analysis and Deconvolution of Barcodes

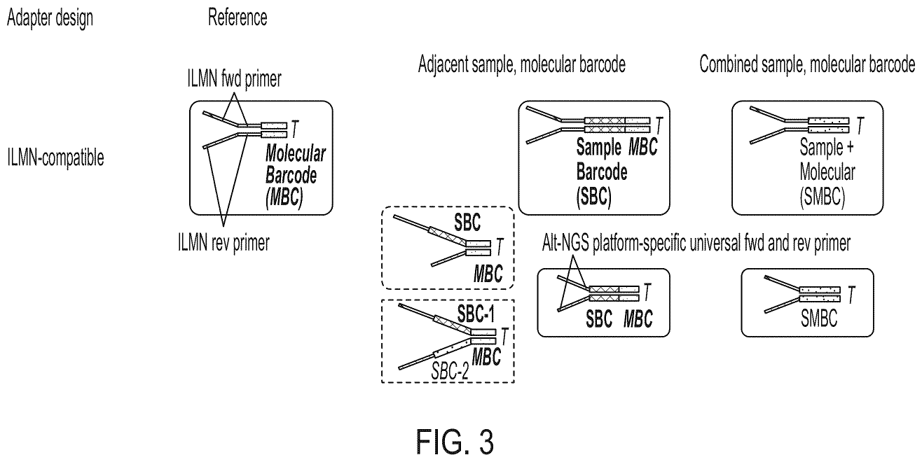

Sequencing of amplification copies of sample nucleic acids flanked by sample and molecular barcodes provided by adapters provides a population of sequencing reads. Sequencing reads typically begin with sequence of upstream molecular and sample barcodes (or combined molecular and sample barcode) followed by sequence of downstream molecular and sometimes a downstream sample barcodes (or combined molecular and sample barcodes). Sequencing reads can be segregated according to their sample of origin by deconvolution of sample barcodes. Sometimes the upstream and downstream sample barcodes on the same sequencing reads are the same, so it is sufficient to look at the upstream sample barcode for deconvolution. Typically the upstream barcode occurring earlier in the sequencing read is the more reliable of the two sample barcode sequences when both are present. But the downstream sample barcode if readable at the end of the sequencing read can be used as a control measure to check the accuracy of the upstream sample barcode (i.e., the two should be the same). When different sample barcodes are incorporated into the respective single-stranded portions of the same adapter as shown in one of the formats in FIG. 3, upstream and downstream sample barcodes are different, and samples can be determined from a combination of the sample barcodes.

Sequencing reads can be segregated into families representing amplification copies of the same original molecule from the molecular barcodes, usually from a combination of upstream and downstream molecular barcodes, and sometimes the sequence of the sample nucleic acid. If unique molecular barcoding is used the molecular barcode or combination of upstream and downstream molecular barcodes is sufficient to indicate family of origin (i.e., all sequencing reads having the same combination of barcodes including complements for the opposing strand are grouped in the same family). If non-unique barcoding is used, then families are identified based on having the molecular barcodes or same combination of molecular barcodes together with a property of sameness among the sequences of sample molecules (such as same start and stop points, or same length) when aligned with a known reference sequence. The sequencing reads within the same family can include sequencing reads from either or both strands of the same original molecule.

The sequencing reads of family members can be compiled to derive consensus nucleotide(s) at specified positions or consensus sequence at some or all positions of a nucleic acid molecule in the original sample. If members of a family include sequencing reads of opposing strands, sequences of one strand can be converted to their complements for purposes of compiling and aligning all sequencing reads to derive consensus nucleotide(s) or sequences. A consensus nucleotide type at a position can be defined as the nucleotide type most frequently occupying that position among aligned sequencing reads. Likewise a consensus sequence can be defined as sequence of such consensus nucleotide types. For a nucleotide type to be called as consensus at a particular position in aligned sequencing reads, it can also be required that the nucleotide type occurs above a threshold frequency level among nucleotide types occupying that position in the aligned sequencing reads. For example, it can be required that the nucleotide type be present at that position in at least 50, 60, 70, 80 or 90% of sequencing reads. It can additionally or alternatively be required that the nucleotide type be present in at least one sequencing read of both strands of an original molecule. It can additionally or alternatively be required that the nucleotide type not be contradicted by more than a threshold number of sequencing reads of one or both strands in which the aligned position is occupied by a different nucleotide type. Consensus deletions or insertions can be identified by similar analyses of representation and/or presence in both strands or substitutions.

Some families may include only a single sequencing read. In this case, this sequence can be taken as the sequence of a nucleic acid in the sample before amplification. Alternatively, families with only a single member sequence can be eliminated from subsequent analysis.

The criteria described above for identifying consensus nucleotides or sequence help filter genuine nucleotide variations from a reference sequence in original sample molecules and variations resulting from amplification or sequencing errors. Nucleic acid variations present in original sample molecules are likely to have greater representation in sequencing reads in general and particularly in sequencing reads of both strands than variations resulting from amplification or sequencing errors and thus be designated as consensus nucleotide types or sequences of such nucleotide types.

Having determined consensus nucleotides and/or consensus sequences within individual families, the results can be compiled to provide an indication of what nucleotide variations are present in a sample compared with a known reference sequence. The known reference sequence can be that of a gene, chromosome or genome among others. Such a compilation can provide an additional filter to distinguish genuine sequence variations from amplification and sequencing errors and provide an indication of the representation or allele frequency of such variations relative to wildtype in a sample. For any position of interest in a reference sequence for a sample (e.g., wildtype human genome sequence), one can determine which families have sequencing reads spanning that position. From those families one can determine a representation of variant nucleotide type, deletion or insertions, if any, and wildtype nucleotide type for that position. A variation can be called out as being present at the position if the number of families including a variant nucleotide type, deletion or insertions exceeds a threshold, or the ratio of families with the variant nucleotide type, deletion or insertion to wildtype exceeds a threshold among other criteria. The ratio of variant nucleotide type, deletion or insertion to wildtype nucleotide type also provides an indication of the representation of the variant nucleotide. Such an analysis can be performed for each nucleotide of interest in a reference sequence corresponding to a particular sample, thus providing a variant profile of that sample. The analysis can be repeated for each sample using families of sequencing reads and their consensus nucleotides or nucleotide sequences derived as discussed above. Thus, each sample can be characterized by a variant nucleotide type profile.

Consensus nucleotides or sequences can also be compared across different sample aliquots subject to treatment resulting in differential substitution of modified and unmodified nucleotides, as in bisulfite analysis. Such analysis indicates which nucleotides in samples molecules are modified, such as by methylation.

Sequence families can also be used to provide an indication of copy number variation (see, e.g., WO2017/106768, WO/2015/100427). The number of families having a consensus sequencing read spanning a particular locus or within a defined window of a genome compared with the number of families mapping to a locus or window elsewhere in the genome, provides a measure of copy number variation, which can arise from either amplification or loss of an allele. Measured numbers of families can be normalized as needed to account for such factors as differences in window size, sequencing coverage or enrichment for different regions of a genome.

### VIII. Applications

The present methods can be used to diagnose presence of conditions, particularly cancer, in a subject, to characterize conditions (e.g., selection of appropriate treatment or staging cancer or determining heterogeneity of a cancer), monitor response to treatment of a condition, effect prognosis risk of developing a condition or subsequent course of a condition.

Various cancers may be detected using the present methods. Cancers cells, as most cells, can be characterized by a rate of turnover, in which old cells die and replaced by newer cells. Generally dead cells, in contact with vasculature in a given subject, may release DNA or fragments of DNA into the blood stream. This is also true of cancer cells during various stages of the disease. Cancer cells may also be characterized, dependent on the stage of the disease, by various genetic aberrations such as copy number variation as well as rare mutations. This phenomenon may be used to detect the presence or absence of cancers individuals using the methods described herein.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like.

Cancers can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, abnormal changes in nucleic acid methylation infection and cancer.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers progress, becoming more aggressive and genetically unstable. Other cancers may remain benign, inactive or dormant. The system and methods of this disclosure can be useful in determining disease progression.

The present methods are also useful in determining the efficacy of particular treatment options. For example, the number of variations detected, irrespective of their precise identity, is a predictor of amenability to immunotherapy because the mutations create neoepitopes that can be subject of immune attack (see e.g., US20200370129).

Other variations or copy number variations indicate suitability of a particular drug. Some examples of such variations are as follows:

TABLE 1

| | Variation | Cancer | Drug |
|---|---|---|---|
| EGFR/ErbB1 | Mutations (e.g. L858R, | NSCLC | gefitinib, erlotinib, afatinib, |
| | ex19del, T790M) | | osimertinib, dacomitinib |
| HER2/ErbB2 | Amplification | Breast | trastuzumab, T-DM1, |
| | | | trastuzumab + pertuzumab, |
| | | | lapatinib, neratinib |
| | Amplification | Esophagogastric | trastuzumab |
| | Point mutations (V659E) | NSCLC | Lapatinib |
| c-Met | ex14 skipping mutations, | NSCLC | crizotinib, capmatinib, |
| | amplification | | savolitinib*, tepotinib |
| RET | Fusion | NSCLC | selpercatinib, pralsetinib, |
| | | | cabozantinib, 3A vandetanib |
| ALK | Fusion | NSCLC | crizotinib, alectinib, ceritinib, |
| | | | lorlatinib brigatinib |
| | Mutations (L1196M, | Soft tissue sarcoma | crizotinib, ceritinib |
| | L1196Q) | | |
| ROS1 | Fusion, mutation | NSCLC | crizotinib, entrectinib |
| NTRK | Fusion | All tumors | larotrectinib, entrectinib |
| c-Kit | Mutations (e.g. | GIST | imatinib, sunitinib, regorafenib, |
| | 449_514mut), deletions | | sorafenib |
| | (e.g. D419del) | | |
| | | Thymic tumors | sunitinib |
| | Mutations (e.g. K642E) | Melanoma | imatinib |
| PDGFR | Mutations (e.g. D842V), | GIST | imatinib, dasatinib |
| | deletions (e.g. | | |
| | C456_N468del) | | |
| | | Leukemia, myelodysplasia | imatinib |
| FGFR1 | Amplification | LSCC | erdafitinib |
| | | NSCLC | AZD4547 |
| FGFR2 | Fusion, mutation | Bladder, | erdafitinib, pemigatinib |
| | | cholangiocarcinoma | |
| | Amplification | Breast | dovitinib |
| FGFR3 | Fusion, mutation | Bladder | erdafitinib |
| RAS | Wild-type | CRC | cetuximab, panitumumab |
| BRAF | Mutations (e.g. V600E) | Melanoma | vemurafenib, dabrafenib, |
| | | | trametinib, trametinib |
| | | NSCLC | dabrafenib + trametinib |
| | | Histiocytosis | cobimetinib |
| | Mutation (V600E) | CRC | encorafenib + cetuximab |
| | Fusions | Ovarian | trametinib, cobimetinib |
| MEK | Mutations | Melanoma, NSCLC, | trametinib, cobimetinib, |
| | | ovarian, histiocytic disorder | selumetinib |
| mTOR | Mutations (e.g. E2014K) | Bladder, RCC | everolimus, temsirolimus |
| AKT | Mutation (E17K) | Breast, ovarian | capivasertib |
| PTEN | Homozygous deletions, | Breast | capivasertib |
| | loss-of-function mutations | | |
| PIK3CA | Mutations | Breast | alpelisib |
| CDK4 | Amplification | Soft tissue sarcoma | palbociclib |
| IDH1 | Mutations | AML, cholangiocarcinoma | ivosidenib |
| IDH2 | Mutations | AML | enasidenib |
| BRCA1/2 and | Mutations (somatic) | Breast | olaparib, talazoparib, rucaparib |
| ATM | | | |
| | Mutations (somatic) | Ovarian, prostate | rucaparib, olaparib |
| ERα | Mutations (e.g. E380Q) | Breast | fulvestrant |
| MSI-H | Not applicable | All | pembrolizumab |
| TML | Not applicable | Multiple tumor types | pembrolizumab, nivolumab |

The present methods can also be used to monitor therapy. For example, a successful treatment can initially be associated with an increase in nucleotide or copy number variations in cell free DNA as cancer cells die and release their DNA to the circulation. This initial increase can be followed by a decrease reflecting fewer if any remaining cancer cells to release their DNA. There can also be a subsequent increase in nucleotide or copy number variations following a period of remission providing an indication of recurrence of the cancer.

The present methods can also be used for detecting genetic variations in conditions other than cancer. Immune cells, such as B cells, undergo copy number variation associated with certain diseases. Clonal expansions can be monitored using copy number variation detection as a measure of disease progression. The present methods may be used to determine or profile rejection activities of the host body, as immune cells attempt to destroy transplanted tissue to monitor the status of transplanted tissue as well as altering the course of treatment or prevention of rejection. Copy number variation or variant nucleotide can be used to determine how a population of pathogens are changing during the course of infection. For example during chronic infections, such as HIV/AIDs or Hepatitis infections, y viruses may change life cycle state and/or mutate into more virulent forms during the course of infection.

The present methods can be used to generate or profile, fingerprint or set of data that is a summation of genetic information derived from different cells in a heterogeneous disease. This set of data may comprise copy number variation and nucleotide variation or both.

The present methods can be used to diagnose, prognose, monitor or observe cancers or other diseases of fetal origin. That is, these methodologies can be employed in a pregnant subject to diagnose, prognose, monitor or observe cancers or other diseases in a unborn subject whose DNA and other nucleic acids may co-circulate with maternal molecules.

### IX. Kits

Any or all of the for performing the above-described methods can be include in a kit. For example, such a kit can include any of the sets of adapters including sample and molecular barcodes. An exemplary kit includes e.g., 2-1000, 10-1000, 100-1000, 10-500, or 100-500 sets of adapters. The sets differ in the sample barcodes and have a common set of molecular barcodes.

### X. Computer Implementation

The present methods can be computer-implemented, such that any or all of the steps described in the specification or appended claims other than wet chemistry steps can be performed in a suitable programmed computer. The computer can be a mainframe, personal computer, tablet, smart phone, cloud, online data storage, remote data storage, or the like. The computer can be operated in one or more locations. A computer program can include codes for performing any of the steps other than wet chemistry steps described in the specification or in the appended claims; for example, code for (d) obtaining sequencing reads of the amplicons, code for segregating the sequence reads according to the sample of origin from a sample barcode portion of the reads and DNA molecule of origin from a molecular barcode portion of the reads to produce for each sample a plurality of families of sequencing reads, the families corresponding to different original molecules, code for calling out genetic variations, if present, for different samples from the plurality of families of sequencing reads for a sample, and code for calling out consensus nucleotides or consensus sequence in a family based on the sequencing reads in that family; and code for calling out genetic variations, if present, for each sample based on the consensus nucleotides and/or consensus sequences present in families for that sample.

The present methods can be implemented in a system (e.g., a data processing system) for analyzing a nucleic acid population. The system can also include a processor, a system bus, a main memory and optionally an auxiliary memory coupled to one another to perform one or more of the steps described in the specification or appended claims, such as the following: obtaining sequencing reads of the amplicons, segregating the sequence reads according to the sample of origin from a sample barcode portion of the reads and DNA molecule of origin from a molecular barcode portion of the reads to produce for each sample a plurality of families of sequencing reads, the families corresponding to different original molecule, calling out genetic variations, if present, for different samples from the plurality of families of sequencing reads for a sample and calling out consensus nucleotides or consensus sequence in a family based on the sequencing reads in that family.

The system can also include a keyboard and/or pointer for providing user input, such as, among other accessories. The system can also include a sequencing apparatus coupled to the memory to provide raw sequencing data.

Various steps of the present methods can utilize information and/or programs and generate results that are stored on computer-readable media (e.g., hard drive, auxiliary memory, external memory, server; database, portable memory device (e.g., CD-R, DVD, ZIP disk, flash memory cards), and the like. For example, information used for and results generated by the methods that can be stored on computer-readable media include control data references sequences, raw sequencing data, sequenced nucleic acids, mutations.

All publications, patents and patent applications, accession numbers, websites and the like mentioned in this specification are incorporated by reference to the same extent as if each individual publication, patent or patent application was so individually denoted. To the extent more different content is associate with an accession number or other reference at different times, the content in effect as of the effective filing date of this application is meant. The effective filing date is the date of the earliest priority application disclosing the accession number in question. Unless otherwise apparent from the context any element, embodiment, step, feature or aspect of the invention can be performed in combination with any other.

### EXAMPLES

### Example 1

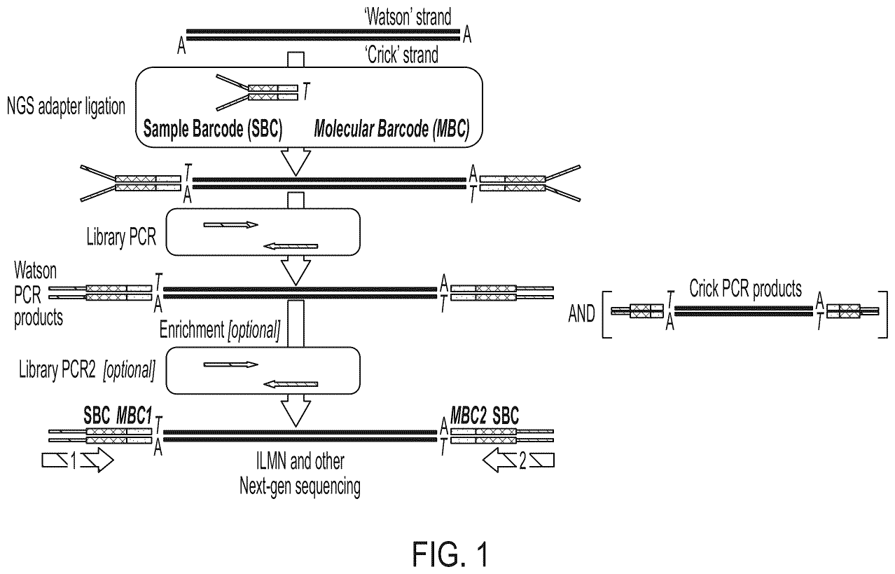

FIG. 1 shows one embodiment of the methods. Sample nucleic acid molecules are provided with a single nucleotide A tail for ligation to T-tailed Y-shaped adapters. The respective strands of the sample molecules are designated Watson and Crick strands. The Y-shaped adapters include a molecular barcode and sample barcode in their double-stranded portion. As shown, the molecular barcode is adjacent the T tail and the sample barcode and molecular barcode together occupy the entire double-stranded portion of the Y-shaped adapter. The single-stranded portions of the Y-shaped adapter contain primer binding sites. In this implementation, the same set of molecular barcodes is used for each sample, and a different sample barcode is used for each sample. Thus, for analyzing a 96 sample batch, 96 sets of adapters each having a different sample barcode and the same set of molecular barcodes (in this example 8 molecular barcodes) is used. After attachment adapters at both ends of sample molecules, the resulting molecules are PCR-amplified with primers binding to sites in the single-stranded portions of the Y-shaped adapters. The amplification products contain sequences from the sample molecules flanked by molecular barcodes flanked by sample barcodes, which are in turn flanked by sequences from the single-stranded portions of the Y-shaped adapters. The orientation of the sequences from the single-stranded portions of the Y-shaped adapters differs in amplification products of the Watson and Crick strands allowing tracing of sequencing reads from the respective strands. The library of sequencing products can undergo enrichment for binding to immobilized oligonucleotides against targeted regions, and optionally further amplification. The resulting amplification products can be sequenced with reads initiating from primer binding sites provided by the originally single-stranded portions of the adapters. Such a sequence read can contain an upstream sample barcode, upstream molecular barcode, sample nucleic acid sequence, downstream molecular barcode and downstream sample barcode in that order. Sequences of strands of amplification products can be read individually or as paired reads in which one read includes moving from upstream to downstream sample barcode, first molecular barcode, sample molecule, second molecular barcode and sample barcode and the paired read includes sample barcode, second molecular barcode, sample molecule, first molecular barcode and sample barcode.

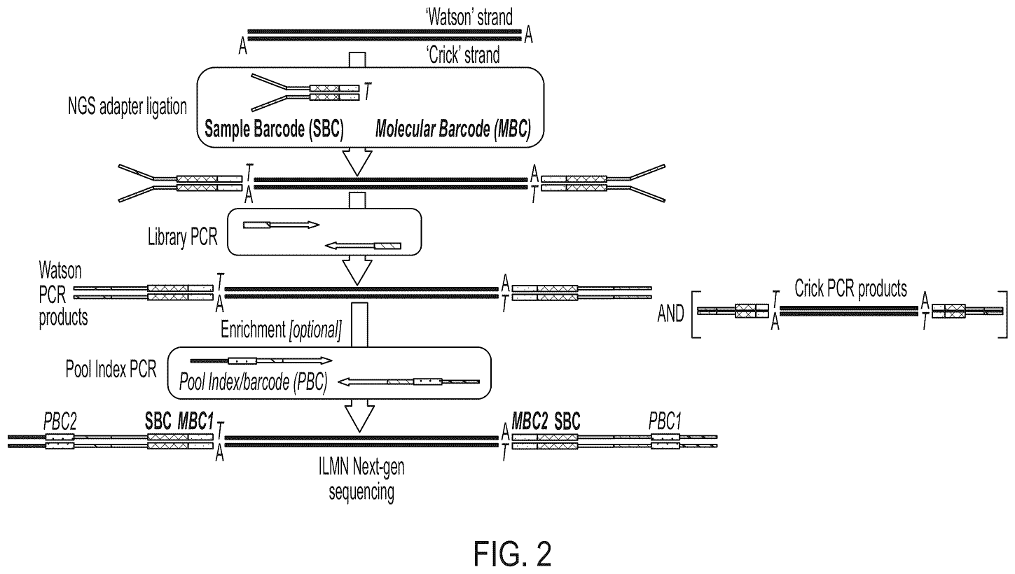

FIG. 2 shows a variation on the method of FIG. 1, in which sample barcodes in the adapter are supplemented by additional sample barcodes as components of primers used in application. This variation is useful when the number of sets of adapters with different sample barcodes is not sufficient for the number of samples to be analyzed. The additional sample barcodes in the primers are referred to in FIG. 2 as pool index barcodes. In FIG. 2, the initial step of attaching Y-shaped adapters to sample nucleic acid molecules is the same as in FIG. 1 except that multiple samples receive the same set of adapters. The samples receiving the same sets of adapters are then distinguished by conducting an amplification step with a primer pair tagged with a pool index (sample) barcodes. As shown both primers of the primer pair have the same pool index barcode. The total number of samples that can be labelled with different sample barcodes is the product of the number of different sample barcodes incorporated into adapters and the number incorporated in primer pairs. For example, if 96 sample barcodes are incorporated into each then 96×96=9216 samples can be labelled. The products of this amplification include a sample nucleic acid molecule flanked by molecular barcodes flanked in turn by sample barcodes deriving from the Y-shaped adapters, flanking in turn by pool index barcodes contributed by primers used in amplification. Using Illumina sequencing, the main library read includes at least a sample barcode, first molecular barcode and sample molecule and optionally second molecular barcode and sample barcode, and a paired library read includes at least a sample barcode, second molecular barcode and sample molecule, and optionally first molecular barcode and sample molecule. The pool index barcodes can be read as separate index reads.

FIG. 3 shows a comparison of three workflows. The left-hand workflow is a reference workflow in which Y-shaped adapters include a molecular barcode in their double-stranded portion and no sample barcode. The sample barcode is added after ligation of adapters to sample nucleic acids as a tail to amplification primers. In the second format (center), Y-shaped adapters include both sample and molecular barcodes as separate sequences with no intervening nucleotides. Sample and molecular barcodes can both be present in the double-stranded portion of Y-shaped adapters or a molecular barcode can be present in the double-stranded portion and a sample barcode in a single-stranded portion. In another format, a molecular barcode is present in the doubled-stranded portion and two sample barcodes are present, one in each single-stranded portion. The third workflow (right) shows a Y-shaped adapter including a combined sample and molecular barcodes. In the first workflow, one set of adapters including 8-105 different molecular barcodes is used. In the second workflow, 96 sets of adapters each containing a different sample barcode, and each containing a set of 8-105 different molecular barcodes is used. In the third workflow 768-10,080 different barcodes are used divided into 96 sets for multiplexing 96 samples are used. The second and third workflows have several advantages relative to the first workflow including less susceptibility to sample contamination, not susceptible to sample barcode hopping between samples, amenability to different sequencing platforms, and amenability to a further layer of sample multiplexing by introducing a further set of sample barcodes as tails to amplification primers. The advantages are summarized in Table 2 below.

TABLE 2

| | | Separate | Combined |
|---|---|---|---|
| | | sample and | sample and |
| | | molecular | molecular |
| | Reference | barcodes | barcodes |
| Barcodes in adapter | Molecular | Sample and | Sample and |
| | | molecular | molecular |
| # Adapter Sequences | 8-105 | 768-10,080 | 768-10,080 |
| # Separate sample | 192 | 0 | 0 |
| barcodes | | | |
| Susceptible to sample | Yes | No | No |
| contamination | | | |
| Alt-NGS platform | No/ | Yes | Yes |
| compatibility | challenging | | |
| Ultra-high sample | Challenging | Yes | Yes |
| multiplex | | | |
| Susceptible to index | Yes | No | No |
| hopping | | | |

FIG. 4 shows a further format in which a Y-shaped adapter includes a molecular barcode in its double-stranded portion and a universal primer binding site formed of unnatural nucleotides in a single-stranded portion to allow introduction of a sample barcode of the same length as the universal primer binding site and contiguous with the molecular barcode in a subsequent amplification step. The single-stranded portions also include primer binding sites for amplification and sequencing. The unnatural nucleotides, such as nitroindole (e.g., 5-nitroindole) and deoxyinosine, can pair with any of the four standard nucleotides in DNA (or RNA). Amplification is performed with a primer pair hybridizing to primer binding sites in the single-stranded portions of the Y-shaped adapters. One of the primers includes a sample barcode at its 3′ end. Amplification with this primer pair introduces the sample barcode in place of the universal primer binding site. Amplification products have a sample molecule flanked by molecular barcodes at each site and a sample barcode at one side. The binding site for the forward primer is at the 3′ of an adapted molecule, such that extension with a sample barcode-containing forward primer occurs first in downstream amplification. Amplification by the reverse primer only occurs on copies made that have sample barcode incorporated. Amplification products can be read from primer binding sites provided by the single-stranded portions of the Y-shaped adapter to yield in one direction a sample barcode followed by an upstream molecular barcode followed by a sample nucleic acid molecule followed by a downstream molecular barcode. In the other direction, the sequence read contains an upstream molecular barcode followed by a sample nucleic acid molecule followed by a downstream molecular barcode followed by a sample barcode.

### Example 2

Directional NGS adapters containing sample indices and molecular barcodes (non-random UMIs) were designed specifically for the NGS sequencing system. The DNA strand of the adapter that ligates to the 5′ end of insert DNA contains, 5′ to 3′: the NGS forward primer sequence (used for PCR amplification and NGS read primer), a first constant sequence region (used in sequencing to calibrate the NGS read), a sample index, a second constant sequence region (used in sequence analysis to identify preceding sample index and proceeding DNA insert sequence), a molecular barcode, and T-tail (other single nucleotide tiles A, C and G can also be used). The DNA strand of the adapter that ligates to the 3′end of the insert DNA contains (5′ to 3′) the reverse complement of the molecular barcode sequence of the other adapter strand, the reverse complement of a portion of the sample index of the other adapter strand and the NGS reverse primer binding site (used in PCR amplification and the sequencing platform workflow). The adapter strands are hybridized, with the molecular barcode containing end of the adapter forming as dsDNA end with a T-tail overhang. Y-adapters are designed, synthesized, and hybridized for each unique molecular barcode and sample index combination used. A set of adapters with different molecular barcode sequences and/or different sample indices are mixed prior NGS library prep in a defined manner and that set of sample/molecular barcode adapters will be assigned to the sample to which they are applied to in library prep.

Library Prep:
1. cfDNA input from a sample is subjected to standard end-repair and A-tailing reaction.
2. The A-tailed reaction is then ligated to a T-tailed adapter set (described above) in standard ligation reaction with T4 DNA ligase.
3. The ligation reaction is cleaned up using a SPRI bead-based method.
4. The NGS libraries are amplified with library universal primers, with the NGS forward and the NGS reverse primer, which hybridizes to the NGS reverse primer binding site sequence in the adapter.
5. The amplified library is cleaned up using a SPRI bead-based method.
6. The amplified library is again amplified with universal primers, the NGS forward primer and the NGS reverse primer with a 5′tail. The 5′tail of the reverse primer makes the resulting PCR product libraries compatible to enter the sequencing platform workflow.
   The full-length targeted library is then processed through the NGS sequencing system and carried through the NGS sequencing workflow.

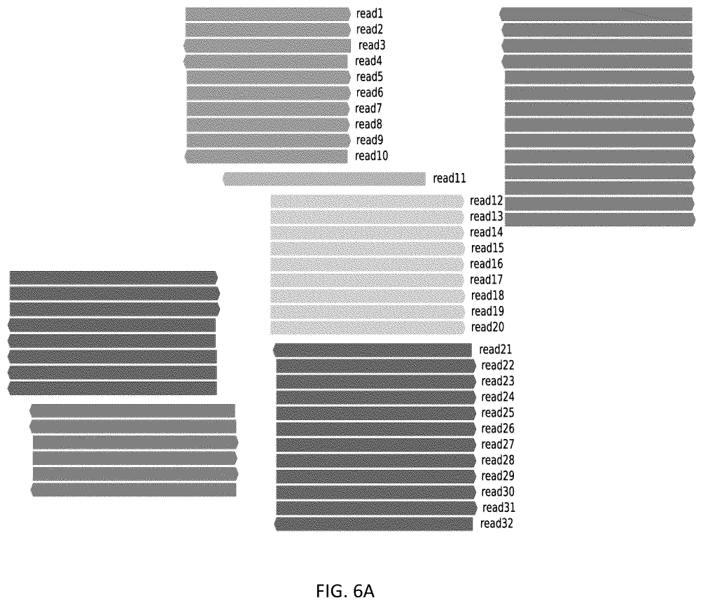

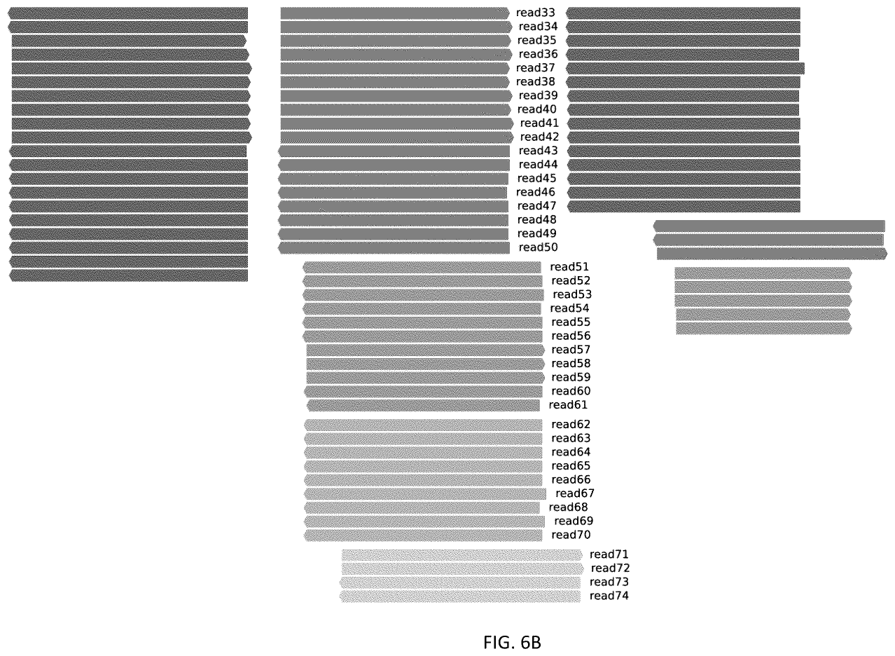

FIG. 5 shows Y-shaped adapters used for analyzing two samples. The adapters include primer binding sites in single-stranded regions and sample and molecular barcodes in double stranded regions. The double-stranded regions are tailed with a T nucleotide to facilitate ligation. The sample barcode is different for samples 1 and 2. In this example, different sets of molecular barcodes are used for samples 1 and 2. The use of different sets of molecular barcodes for different samples is for purposes of illustration, and in practice the same set of molecular barcodes can be used for each of the samples. FIGS. 6A, B and Table 3 show a collection of sequencing reads, for which the sequence has been split into sample barcode, molecular barcodes, and the insert, and where the insert sequence has been aligned to the human reference genome HG19.

FIGS. 6A, B shows the alignment of the sequencing reads to the genome. Table 3 shows a subset of reads with their sample barcodes, molecular barcodes and alignment coordinates. Reads 1-32 are assigned to sample 1 based on their sample barcode. Reads 1-10 are grouped into a single family (family 1) because they:
were assigned to the same sample,
have the same pair of molecule barcodes,
their start coordinates are within 4 bp of each other,
their end coordinates are within 4 bp of each other.

Similarly, reads 12-20 were grouped into family 3; reads 21-32 were grouped into family 4. Read 11 could not be grouped with any other reads in sample 1, therefore it was assigned its own family 2. Reads 33-74 are assigned to sample 2 based on their sample barcode. Reads 33-50 were grouped into family 5; reads 51-61 were grouped into family 6; reads 62-70 were grouped into family 7; and reads 71-74 were grouped into family 8. All above conditions were required to be satisfied to group reads into a common family. For example, reads 11 could not be grouped with reads 1-10 despite having the same sample and same molecule barcodes, but the start and end coordinates were too distant. Similarly reads 51-61 could not be grouped with reads 62-70 despite having the same sample, and very similar start and end coordinates, because the molecular barcodes were different.

TABLE 3

| | | Start | | End | | | |
|---|---|---|---|---|---|---|---|
| | Sample | Molecule | Start | Molecule | End | Sample | Family |
| Read ID | BC | BC | Coordinate | BC | Coordinate | Assignment | Assignment |
| read1 | SB1 | MB1 | 1:30276939 | MB1 | 1:30277080 | sample1 | family1 |
| read2 | SB1 | MB1 | 1:30276939 | MB1 | 1:30277080 | sample1 | family1 |
| read3 | SB1 | MB1 | 1:30276939 | MB1 | 1:30277082 | sample1 | family1 |
| read4 | SB1 | MB1 | 1:30276939 | MB1 | 1:30277079 | sample1 | family1 |
| read5 | SB1 | MB1 | 1:30276940 | MB1 | 1:30277080 | sample1 | family1 |
| read6 | SB1 | MB1 | 1:30276940 | MB1 | 1:30277080 | sample1 | family1 |
| read7 | SB1 | MB1 | 1:30276940 | MB1 | 1:30277079 | sample1 | family1 |
| read8 | SB1 | MB1 | 1:30276940 | MB1 | 1:30277079 | sample1 | family1 |
| read9 | SB1 | MB1 | 1:30276940 | MB1 | 1:30277080 | sample1 | family1 |
| read10 | SB1 | MB1 | 1:30276940 | MB1 | 1:30277079 | sample1 | family1 |
| read11 | SB1 | MB1 | 1:30276973 | MB1 | 1:30277147 | sample1 | family2 |
| read12 | SB1 | MB2 | 1:30277013 | MB1 | 1:30277179 | sample1 | family3 |
| read13 | SB1 | MB2 | 1:30277013 | MB1 | 1:30277179 | sample1 | family3 |
| read14 | SB1 | MB2 | 1:30277013 | MB1 | 1:30277179 | sample1 | family3 |
| read15 | SB1 | MB2 | 1:30277013 | MB1 | 1:30277180 | sample1 | family3 |
| read16 | SB1 | MB2 | 1:30277013 | MB1 | 1:30277179 | sample1 | family3 |
| read17 | SB1 | MB2 | 1:30277013 | MB1 | 1:30277179 | sample1 | family3 |
| read18 | SB1 | MB2 | 1:30277013 | MB1 | 1:30277180 | sample1 | family3 |
| read19 | SB1 | MB2 | 1:30277013 | MB1 | 1:30277180 | sample1 | family3 |
| read20 | SB1 | MB2 | 1:30277013 | MB1 | 1:30277180 | sample1 | family3 |
| read21 | SB1 | MB1 | 1:30277017 | MB1 | 1:30277187 | sample1 | family4 |
| read22 | SB1 | MB1 | 1:30277018 | MB1 | 1:30277189 | sample1 | family4 |
| read23 | SB1 | MB1 | 1:30277018 | MB1 | 1:30277189 | sample1 | family4 |
| read24 | SB1 | MB1 | 1:30277018 | MB1 | 1:30277189 | sample1 | family4 |
| read25 | SB1 | MB1 | 1:30277018 | MB1 | 1:30277189 | sample1 | family4 |
| read26 | SB1 | MB1 | 1:30277018 | MB1 | 1:30277189 | sample1 | family4 |
| read27 | SB1 | MB1 | 1:30277018 | MB1 | 1:30277189 | sample1 | family4 |
| read28 | SB1 | MB1 | 1:30277018 | MB1 | 1:30277189 | sample1 | family4 |
| read29 | SB1 | MB1 | 1:30277018 | MB1 | 1:30277189 | sample1 | family4 |
| read30 | SB1 | MB1 | 1:30277018 | MB1 | 1:30277189 | sample1 | family4 |
| read31 | SB1 | MB1 | 1:30277018 | MB1 | 1:30277190 | sample1 | family4 |
| read32 | SB1 | MB1 | 1:30277018 | MB1 | 1:30277188 | sample1 | family4 |
| read33 | SB2 | MB4 | 1:30276960 | MB3 | 1:30277125 | sample2 | family5 |
| read34 | SB2 | MB4 | 1:30276960 | MB3 | 1:30277127 | sample2 | family5 |
| read35 | SB2 | MB4 | 1:30276960 | MB3 | 1:30277126 | sample2 | family5 |
| read36 | SB2 | MB4 | 1:30276960 | MB3 | 1:30277127 | sample2 | family5 |
| read37 | SB2 | MB4 | 1:30276960 | MB3 | 1:30277125 | sample2 | family5 |
| read38 | SB2 | MB4 | 1:30276960 | MB3 | 1:30277125 | sample2 | family5 |
| read39 | SB2 | MB4 | 1:30276960 | MB3 | 1:30277127 | sample2 | family5 |
| read40 | SB2 | MB4 | 1:30276960 | MB3 | 1:30277126 | sample2 | family5 |
| read41 | SB2 | MB4 | 1:30276960 | MB3 | 1:30277128 | sample2 | family5 |
| read42 | SB2 | MB4 | 1:30276960 | MB3 | 1:30277128 | sample2 | family5 |
| read43 | SB2 | MB4 | 1:30276960 | MB3 | 1:30277127 | sample2 | family5 |
| read44 | SB2 | MB4 | 1:30276960 | MB3 | 1:30277127 | sample2 | family5 |
| read45 | SB2 | MB4 | 1:30276960 | MB3 | 1:30277126 | sample2 | family5 |
| read46 | SB2 | MB4 | 1:30276960 | MB3 | 1:30277125 | sample2 | family5 |
| read47 | SB2 | MB4 | 1:30276960 | MB3 | 1:30277126 | sample2 | family5 |
| read48 | SB2 | MB4 | 1:30276960 | MB3 | 1:30277126 | sample2 | family5 |
| read49 | SB2 | MB4 | 1:30276960 | MB3 | 1:30277126 | sample2 | family5 |
| read50 | SB2 | MB4 | 1:30276960 | MB3 | 1:30277127 | sample2 | family5 |
| read51 | SB2 | MB4 | 1:30276978 | MB3 | 1:30277150 | sample2 | family6 |
| read52 | SB2 | MB4 | 1:30276978 | MB3 | 1:30277151 | sample2 | family6 |
| read53 | SB2 | MB4 | 1:30276978 | MB3 | 1:30277152 | sample2 | family6 |
| read54 | SB2 | MB4 | 1:30276978 | MB3 | 1:30277150 | sample2 | family6 |
| read55 | SB2 | MB4 | 1:30276978 | MB3 | 1:30277151 | sample2 | family6 |
| read56 | SB2 | MB4 | 1:30276978 | MB3 | 1:30277151 | sample2 | family6 |
| read57 | SB2 | MB4 | 1:30276979 | MB3 | 1:30277151 | sample2 | family6 |
| read58 | SB2 | MB4 | 1:30276979 | MB3 | 1:30277151 | sample2 | family6 |
| read59 | SB2 | MB4 | 1:30276979 | MB3 | 1:30277151 | sample2 | family6 |
| read60 | SB2 | MB4 | 1:30276979 | MB3 | 1:30277151 | sample2 | family6 |
| read61 | SB2 | MB4 | 1:30276981 | MB3 | 1:30277149 | sample2 | family6 |
| read62 | SB2 | MB3 | 1:30276979 | MB4 | 1:30277151 | sample2 | family7 |
| read63 | SB2 | MB3 | 1:30276979 | MB4 | 1:30277151 | sample2 | family7 |
| read64 | SB2 | MB3 | 1:30276979 | MB4 | 1:30277151 | sample2 | family7 |
| read65 | SB2 | MB3 | 1:30276979 | MB4 | 1:30277151 | sample2 | family7 |
| read66 | SB2 | MB3 | 1:30276979 | MB4 | 1:30277151 | sample2 | family7 |
| read67 | SB2 | MB3 | 1:30276979 | MB4 | 1:30277154 | sample2 | family7 |
| read68 | SB2 | MB3 | 1:30276979 | MB4 | 1:30277149 | sample2 | family7 |
| read69 | SB2 | MB3 | 1:30276979 | MB4 | 1:30277153 | sample2 | family7 |
| read70 | SB2 | MB3 | 1:30276979 | MB4 | 1:30277151 | sample2 | family7 |
| read71 | SB2 | MB4 | 1:30277005 | MB4 | 1:30277179 | sample2 | family8 |
| read72 | SB2 | MB4 | 1:30277005 | MB4 | 1:30277180 | sample2 | family8 |
| read73 | SB2 | MB4 | 1:30277005 | MB4 | 1:30277179 | sample2 | family8 |
| read74 | SB2 | MB4 | 1:30277005 | MB4 | 1:30277179 | sample2 | family8 |

1. A method of sequencing populations of DNA molecules in multiple samples, comprising:
(a) ligating a population of DNA molecules from a first sample to a first set of adapters, such that molecules of the population are flanked by an adapter on each side, wherein each adapter includes primer binding sites, and a molecular barcode varying among members of the set of adapters and a sample barcode that is the same among members of the set of adapters, wherein the molecular and sample barcodes are situated in the adapter such that a sequencing read initiating from one of the primer binding site of the adapter includes sequence of the sample and molecular barcodes followed by sequence of a DNA molecule of the first sample;
(b) repeating step (a) on populations of DNA molecules from one or more further samples, except that the populations of DNA molecules from each sample are ligated to different set of adapters, wherein the sample barcode varies among the different sets of adapters;
(c) amplifying the DNA molecules flanked by adapters to generate amplicons, each amplicon comprising a DNA molecule flanked by barcodes of the adapters on each side, flanked by primer binding sites of the adapters on each side;
(d) obtaining sequencing reads of the amplicons, wherein each sequencing read is initiated from one of the sequencing primer binding sites provided by the adapters; and
(e) segregating the sequence reads according to the sample of origin from a sample barcode portion of the reads and DNA molecule of origin from a molecular barcode portion of the reads to produce for each sample a plurality of families of sequencing reads, the families corresponding to different original molecules.

2. The method of claim 1 further comprising (f) calling out genetic variations, if present, for different samples from the plurality of families of sequencing reads for a sample.

3. The method of claim 2, wherein step (f) comprises
for some or all of the families, calling out consensus nucleotides or consensus sequence in a family based on the sequencing reads in that family; and
calling out genetic variations, if present, for each sample based on the consensus nucleotides and/or consensus sequences present in families for that sample.

4. The method of any preceding claim, further comprising pooling the adapted DNA molecules from the different samples after step (b) and before step (c).

5. The method of any one of claims 1-3, wherein step (c) is performed separately for different samples with a primer containing a pool index, and the method further comprises pooling amplification products after step (c).

6. The method of any preceding claim, wherein the same set of molecular barcodes is used for each set of adapters.

7. The method of any preceding claim, wherein the sample barcode portion and the molecular barcode portion are contiguous sequences.

8. The method of any preceding claim, wherein each adapter has two sample barcodes.

9. The method of any preceding claim, wherein the sequencing reads in at least some of the families include sequencing reads of both strands of the same original molecule.

10. The method of any preceding claim, wherein segregation into families is based on molecular barcode sequences and sequences of the molecules of the population.

11. The method of any preceding claim, wherein the adapters comprise one or more double-stranded portions and one or more single-stranded portions.

12. The method of claim 11, wherein the adapters are Y-shaped adapters comprising two strands duplexed in a double-stranded portion and unduplexed in single-stranded portions.

13. The methods of claim 11, wherein the adapters are stem-loop adapters, the stem providing a double-stranded portion, and the loop comprising two single-stranded portions separated by a uracil or deoxyuridine residue.

14. The method of claim 11, wherein the adapters are bubble adapters comprising two strands, forming unduplexed single-stranded portions flanked by duplexed double-stranded portions.

15. The method of any preceding claim, wherein the primer binding sites are in the single-stranded portions of the adapters.

16. The method of any preceding claim, wherein the molecular barcode of each adapter is in a double-stranded portion of the adapter.

17. The method of claim 16, wherein the molecular barcode of each adapter is flush with the free end of the double-stranded portion of the adapter containing the molecular barcode portion.

18. The method of any preceding claim, wherein the sample barcode and the molecular barcode are separate but contiguous sequences.

19. The method of claim 18, wherein the sample barcode and the molecular barcode are separate but contiguous sequences within the double-stranded portion of the adapters.

20. The method of claim 19, wherein the double-stranded portion of the adapters consists of the sample barcode and the molecular barcode.

21. The method of any one of claims 1-18, wherein the molecular barcode is in a double-stranded portion and the sample barcode or sample barcodes is/are within one or both of the single-stranded portions of the adapters.

22. The method of claim 21, wherein the molecular barcode is in the double-stranded portion and two sample barcode are respectively within the single-stranded portions of the adapters.

23. The method of any preceding claim, wherein the DNA molecules are cell-free DNA molecules.

24. The method of any preceding claim, wherein the molecular barcodes non-uniquely label the DNA molecules in the sample.

25. The method of claim 24, wherein the number of different pairwise combinations of molecular barcodes is less than 1/104 of the number of DNA molecules.

26. The method of any preceding claim, wherein the amplification is performed with primers binding to the primer binding sites.

27.-70. (canceled)
